(19) United States
(12) Patent Application Publication
(10) Pub. No.: US 2024/0169440 A1
(43) Pub. Date: May 23, 2024

(54) PHARMACEUTICAL CLAIM NETWORK ROUTING SYSTEM

(71) Applicant: MedImpact Healthcare Systems, Inc., San Diego, CA (US)

(72) Inventors: Timothy Rourke, San Diego, CA (US); William J. Barre, San Diego, CA (US)

(21) Appl. No.: 17/992,654

(22) Filed: Nov. 22, 2022

Related U.S. Application Data
(63) Continuation of application No. 15/439,820, filed on Feb. 22, 2017.
Application No. 15/439,820 is a continuation of application No. 14/209,853, filed on Mar. 13, 2014, now abandoned.
(60) Provisional application No. 61/786,076, filed on Mar. 14, 2013.

(51) Int. Cl. G06Q 40/08; G06Q 30/02

(52) U.S. Cl. CPC G06Q 40/08; G06Q 30/0206

(57) ABSTRACT

Systems and methods for routing pharmaceutical claims in a network are provided. The system includes a network interface that includes a receiver and a transmitter. The receiver is configured to receive an electronic prescription and patient information over a communications network and the transmitter configured to prepare data for transmission over the communications network. The system includes at least one processor communicatively coupled to the network interface. The processor is configured for receiving, using the at least one processor and via the communications network, the electronic prescription. The processor is further configured for accessing a database of pricing data communicatively coupled to the communications network. The processor is configured to select a suggested pharmacy accessible to the patient by evaluating the plurality of participating pharmacies. The processor is configured to send the prescription electronically to a computing system of the suggested pharmacy for fulfillment.

### CROSS REFERENCE TO RELATED APPLICATION

This application claims the benefit of and priority to and is a continuation of U.S. patent application Ser. No. 15/439,820, filed on Feb. 22, 2017, which is a continuation of U.S. patent application Ser. No. 14/209,853, filed on Mar. 13, 2014, which claims priority to and benefit of U.S. Provisional Application 61/786,076, filed on Mar. 14, 2013, the entirety of each of which is incorporated by reference herein.

### BACKGROUND

### Technical Field

The present technology relates to systems and methods for routing pharmaceutical claims over a network. More particularly, the present technology relates to computer architecture and operating methods for routing claim switches over a computer network.

### Description of the Related Art

Computing systems can include a processor, a memory, a storage device, and input/output devices. The processor, the memory, the storage device, and the input/output devices can be interconnected via a system bus. The processor is capable of processing instructions for execution within the computing system. Such executed instructions can implement one or more components of, for example, a cloud platform. The computing system may include input/output devices that can provide input/output operations for a network device. For example, the input/output device can include Ethernet ports or other networking ports to communicate with one or more wired and/or wireless networks (e.g., a local area network (LAN), a wide area network (WAN), the Internet) or hardware or software implemented communications switches placed within the networked environment.

In conventional network systems, computing systems select one repository for storing data in one logical database across a network. Conventional computing systems typically retrieve data from and store data on their own systems. A computing system may remotely access one of a plurality of server systems across a network that might in turn access the database system. Data retrieval from the system might include the issuance of a request from the user system to the database system. The database system may process the request for information and send to the user system information relevant to the request. The rapid and efficient retrieval of accurate information is critical to routing claims over a computer network environment in which a plurality of computing systems access and retrieve data from a plurality of databases coupled to a communications network.

Many computing systems coordinate and/or generate claims over a network to have patient pharmaceutical costs covered through prescription benefit plans (“plans”). These plans are generally offered to patients through health maintenance organizations, employer groups, and government entities (“payers”). Under such plans, a patient receives an electronic prescription for a medication from his or her physician and submits it to a pharmacy computing system to be filled. The pharmacy computing system checks to see that the patient is a member of a plan with which the pharmacy has an agreement and that the medication and dosage prescribed are within the approved scope of the plan agreement. Upon verification of these requirements, the pharmacy computing system instructs the dispensing of the medication to the patient. The patient pays the pharmacy a copayment amount (“copay”), which is less than the normal cost of the medication. The pharmacy computing system receives the balance of the payment for the medication and its dispensing services from the plan, which is managed by a prescription benefit manager (“PBM”) with whom the payer has agreed to manage the plan. The PBM invoices the payer (i.e., the PBM's customer) for the patient's transaction, along with a charge for the agreed fee. From the funds paid by the payer, the PBM pays the pharmacy's balance due. In such a computing system, payers typically turn to PBMs to manage costs. Because the cost of medications is so high and is such a large component of medical care costs generally, there is an on-going effort on the part of PBMs and payers to seek ways to control medication costs. As all healthcare costs continue to rise, there is also an ongoing need for more controlled management and costs of patients' other healthcare needs.

### SUMMARY

The systems and methods described herein each have several aspects, no single one of which is solely responsible for its desirable attributes. Without limiting the scope of this disclosure as expressed by the claims that follow, the more prominent features will now be discussed briefly. After considering this discussion, and particularly after reading the section entitled “Detailed Description” one will understand how the sample features described herein provide for a more comprehensive system having several advantages over current prescription drug-fulfillment systems.

The systems and methods described herein relate to a total healthcare needs fulfillment system. The healthcare needs fulfillment system of various embodiments is a central computing system which coordinates with physicians, pharmacies, mail-order pharmacies, specialty providers, and patients in order to provide some or all of the medical goods needed by a patient. In some embodiments, medical goods include, but are not limited to, one or more of the following: prescription drugs, biotechnology therapies, over-the-counter medications, dietary supplements, disposable medical supplies, and durable medical equipment. The total healthcare needs fulfillment system of some embodiments also coordinates with physicians, specialty providers, and patients to provide services such as, for example, imaging services. In some embodiments, for example, the healthcare needs fulfillment system is designed to receive a prescription script from a physician prescribing a medical good or service for a patient. The system of some embodiments performs checks to ensure that the prescribed good or service is appropriate for the patient. In some embodiments, the healthcare needs fulfillment system identifies one or more suggested pharmacies or suppliers for filling a patient's prescription. A pharmacy or supplier may be suggested by the system based on one or more criteria, for example, based on the price charged by the pharmacy/supplier for the prescribed good or service, the proximity of the pharmacy/supplier to the patient, the performance rating of the pharmacy/supplier and/or the expertise of the pharmacy/supplier.

In some embodiments, the healthcare needs fulfillment system facilitates communication with the patient, such as, for example, through a call center, to notify the patient when a better pharmacy/supplier is available for providing the patient's prescribed good or service. In some embodiments, determining whether a better pharmacy/supplier exists involves applying a rule set that factors in one or more ranking criteria, such as for example, any of the criteria listed in the preceding paragraph. In some embodiments, the healthcare needs fulfillment system provides content, applications, and/or web-based interfaces designed to push healthcare management downstream to patients. The healthcare needs fulfillment system of some embodiments may, for example, facilitate communication with the patient to alert the patient of potential drug interactions or health and wellness tips and reminders. The system of some embodiments provides personalized content related to medical and health topics of relevance to a particular patient to empower the patient. In some embodiments, the healthcare needs fulfillment system provides a web-based interface through which a patient can easily view all of the patient's prescribed medical goods and services—even if several pharmacies and suppliers are providing the various goods and services. In some such embodiments, a patient logged into a web portal can track the status of each prescribed good and service and provide financial information to facilitate the automatic payment of copays.

These are just some of the system's potential features. Any particular system may have some or all of these features or additional or alternative features. Some such additional and alternative features of the system will become apparent in the description that follows. The various features of the system are provided to fulfill a patient's healthcare needs while minimizing the patient's healthcare management burden.

One aspect of the disclosure provides a method of fulfilling and managing prescriptions. The method of some embodiments includes: receiving a prescription in electronic form, wherein the prescription comprises a pharmaceutical drug name and a dosage; receiving patient information in electronic form, wherein the patient information comprises an identifier linking a patient to a prescription benefit plan if applicable; accessing a database of drug pricing data comprising current drug prices at a plurality of participating pharmacies; determining a suggested pharmacy based at least in part on the pharmaceutical drug name, the dosage, the prescription benefit plan if applicable, and the current drug pricing data; and sending the electronic prescription to a selected pharmacy for fulfillment.

In some embodiments of the method, the suggested pharmacy is also determined based, at least in part, on the location of the plurality of participating pharmacies. In some embodiments, the suggested pharmacy is automatically chosen to be the selected pharmacy. In other embodiments, identifying a suggested pharmacy includes: identifying a plurality of suggested pharmacy options, providing a list of the suggested pharmacy options to the user in a selectable format, and receiving an input from the user indicating the selected pharmacy. In some embodiments, the plurality of suggested pharmacy options comprises a mail-order option and one or more pharmacies. In some embodiments, the prescription and patient information is provided in electronic form by a user. In some such embodiments, the user is the patient or physician healthcare provider of the patient. In other embodiments, the prescription and patient information are sent by a user in a non-electronic format, and are later received by the system in electronic format upon uploading by an individual or computer. In some embodiments, the identifier is a patient-specific identification code or username linking the patient to a patient-specific profile, which stores patient-specific information, including at least the patient's: prescription benefit plan, birthdate, name, address, and current prescriptions. The patient-specific profile of some embodiments stores financial account information for the patient. In some such embodiments, the method further includes deducting payment from the financial account information when the electronic prescription is received, adjudicated, or fulfilled by the selected pharmacy.

In some embodiments, the method further includes tracking the electronic prescription and updating a prescription status that is viewable by the user when the selected pharmacy receives the electronic prescription, when the selected pharmacy adjudicates the electronic prescription, and when the selected pharmacy fulfills the electronic prescription.

In some embodiments, the method further includes receiving a question from the patient, identifying a category to which the question pertains, and directing the question to an appropriate resource. In such embodiments, an order status question or a payment question is directed to the selected pharmacy, a benefits question is directed to the patient's pharmacy benefits manager, and a health question is directed to a professional health services representative or healthcare provider.

In some embodiments of the method, the current drug prices are updated periodically to reflect pricing data received from a plurality of participating pharmacies during a bidding process. In such embodiments, the bidding process includes: providing various pharmacies with access to participate in bidding covering a next pricing cycle, and receiving bids for the next pricing cycle from a plurality of participating pharmacies.

Another aspect of the disclosure includes a system for fulfilling and managing prescriptions. The system of various embodiments includes: a receiver configured to receive an electronic prescription and patient information from a user via a web-based interface, wherein the electronic prescription comprises a pharmaceutical drug name and a dosage, and the patient information comprises an identifier linking a patient to a prescription benefit plan if applicable; a database of drug pricing data comprising current drug prices at a plurality of participating pharmacies; a processor configured to determine a suggested pharmacy based at least in part on the pharmaceutical drug name, the dosage, the prescription benefit plan, and the current drug pricing data; and a transmitter configured to send the electronic prescription via a web-based interface to a selected pharmacy for fulfillment.

In some embodiments, the system further includes a memory configured to store a patient-specific profile, which comprises patient-specific information, including at least the patient's: prescription benefit plan, birthdate, name, address, and current prescriptions. In some embodiments, the system further includes a memory configured to store pharmacy-specific information, including at least a name and an address for each pharmacy. In some such embodiments, the memory comprises a web-accessible database.

A further aspect of the disclosure is directed to a method of fulfilling and managing prescriptions. The method includes: receiving a processed claim record from a payor, PBM, or claim aggregator, wherein the processed claim record identifies a fulfilling pharmacy, a prescription drug, a patient, and an address of the patient; generating an eligible claim switch file; sending the eligible claim switch file to a pharmacy evaluator; receiving a report from the pharmacy evaluator identifying a suggested pharmacy, wherein the pharmacy evaluator identifies a suggested pharmacy by evaluating a plurality of pharmacies at least in part on proximity to the patient and price of the prescription drug; determining if the patient is a target patient, wherein a patient is a target patient if the fulfilling pharmacy is not the suggested pharmacy; and sending a list including the target patient to a caller for contacting to determine if the target patient wants to transfer the prescription to the suggested pharmacy.

In some embodiments of the method, the suggested pharmacy includes a plurality of suggested pharmacy options. In some embodiments, the method further includes calling the target patient to determine if the target patient would like to transfer the prescription to the suggested pharmacy.

In some embodiments, the method further includes: receiving a notification from the caller when the target patient authorizes a transfer to a new pharmacy, and contacting the new pharmacy to transfer the prescription for the patient, wherein the new pharmacy is the suggested pharmacy or one of the plurality of suggested pharmacy options. In some embodiments, the pharmacy evaluator is an outside vendor or operated by an outside vendor. Additionally or alternatively, in some embodiments, the pharmacy evaluator is a server or computer. In some embodiments, the caller is an outside vendor or operated by an outside vendor. Additionally or alternatively, in some embodiments, the caller is a person or computer.

Furthermore, an aspect of the disclosure is directed to a system for fulfilling and managing prescriptions in which the system includes: a receiver configured to receive a processed claim record from a payor, PBM, or claim aggregator via a web-based interface, wherein the processed claim record identifies a fulfilling pharmacy, a prescription drug, a patient, and an address of the patient; a processor configured to generate an eligible claim switch file; and a transmitter configured to send the eligible claim switch file to a pharmacy evaluator. In some embodiments, the receiver is further configured to receive a report from the pharmacy evaluator identifying a suggested pharmacy, the processor is further configured to determine if the patient is a target patient (wherein a patient is a target patient if the fulfilling pharmacy is not the suggested pharmacy), and the transmitter is further configured to send a list including the target patient to a caller for contacting to determine if the target patient wants to transfer the prescription to the suggested pharmacy.

The foregoing is a summary and thus contains, by necessity, simplifications, generalization, and omissions of detail; consequently, those skilled in the art will appreciate that the summary is illustrative only and is not intended to be in any way limiting. Other aspects, features, and advantages of the systems, methods, devices, and/or processes described herein will become apparent in the teachings that follow.

### BRIEF DESCRIPTION OF THE DRAWINGS

The above-mentioned aspects, as well as other features, aspects, and advantages of the present technology will now be described in connection with various embodiments, with reference to the accompanying drawings. The illustrated embodiments, however, are merely examples and are not intended to be limiting.

FIG. 1A is a block diagram of one embodiment of a healthcare needs fulfillment system, which depicts the system participants and interactions between the participants.

FIG. 1B is a functional block diagram of one embodiment of a healthcare needs fulfillment system.

FIG. 2 is a flowchart illustrating one embodiment of a method for fulfilling and managing prescriptions performed by a healthcare needs fulfillment system.

FIG. 3 is a flowchart illustrating another embodiment of a method for fulfilling and managing prescriptions performed by a healthcare needs fulfillment system.

FIG. 4 is a flowchart illustrating another embodiment of a method for fulfilling and managing prescriptions performed by a healthcare needs fulfillment system.

FIG. 5A is a flowchart illustrating an embodiment of a method for identifying a preferred provider of a pharmaceutical drug, the method performed by a healthcare needs fulfillment system.

FIG. 5B is a flowchart illustrating another embodiment of a method for identifying a preferred provider of a pharmaceutical drug, the method performed by a healthcare needs fulfillment system.

FIG. 6 is a flowchart illustrating an embodiment of a method for answering patients' questions, the method performed by a healthcare needs fulfillment system.

### DETAILED DESCRIPTION OF CERTAIN EMBODIMENTS

In the following detailed description, reference is made to the accompanying drawings, which form a part of the present disclosure. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the Figures, can be arranged, substituted, combined, and designed in a wide variety of different configurations, all of which are explicitly contemplated and form part of this disclosure. For example, a system or apparatus may be implemented or a method may be practiced using any number of the aspects set forth herein. In addition, such a system or apparatus may be implemented or such a method may be practiced using other structure, functionality, or structure and functionality in addition to or other than one or more of the aspects set forth herein.

Similarly, methods disclosed herein may be performed by one or more computer processors configured to execute instructions retrieved from a computer-readable storage medium. A computer-readable storage medium stores information, such as data or instructions, for some interval of time, such that the information can be read by a computer during that interval of time. Examples of computer-readable storage media are memory, such as random access memory (RAM), and storage, such as hard drives, optical discs, flash memory, floppy disks, magnetic tape, paper tape, punch cards, and Zip drives.

For convenience of description and ease of understanding, the discussion and examples provided herein are largely directed to systems and methods for fulfilling and managing pharmaceutical prescriptions. However, one of skill in the art will understand that the content of the present application applies equally to other healthcare needs, and all such applications are expressly contemplated and hereby form part of this disclosure. For example, each of the systems and methods described herein may be used for fulfillment and management of orders for any healthcare good or service prescribed or recommended to a patient, such as biologic therapeutics, supplements, vitamins, over-the-counter medications, durable medical goods, and/or disposable medical supplies.

### Introduction

The systems and methods described herein relate to a total healthcare needs fulfillment system. The healthcare needs fulfillment system of various embodiments is a central portal which coordinates with physicians, pharmacies, mail-order pharmacies, specialty providers, and patients in order to address all of a patient's healthcare needs, while minimizing the burden on the patient. The healthcare needs fulfillment system of various embodiments is designed to reduce healthcare costs and improve patient care and compliance. In some embodiments, the healthcare needs fulfillment system is particularly designed to reduce the cost of prescriptions drugs and improve patient adherence to prescribed prescription drug regimens.

Medication usage is commonly differentiated between acute care usage, which is short term (30 days or less) administration to treat immediate illnesses or conditions, and maintenance usage, which is long term (more than 30 days) treatment of chronic illnesses or conditions such as hypertension, high cholesterol levels, arthritis, neurology conditions and the like. Maintenance medication dispensing and usage represents a major health care cost (on the order of 75% of prescription costs for many plans, especially due to aging of the American population), and therefore, control of maintenance prescription costs is a principal function of the prescription benefit plans and the PBMs that manage them. Dispensing pharmacies are normally of two types: pharmacies (which are local neighborhood businesses where the patient appears in person, can meet with a pharmacist, orders his/her medication, and can usually leave a few minutes later with the dispensed medication in hand) and mail order pharmacies (which are large facilities, usually not open to personal visits from individual patients, but from which a patient's medication order received by mail or through the internet is subsequently filled and dispensed to the patient via mail or courier service). It is normally recognized by the industry that acute care prescriptions are dispensed primarily by pharmacies, since the patient frequently needs the medication immediately and cannot accept the multiday delay inherent in submitting and dispensing prescription medications from the mail order pharmacies.

On the other hand, PBMs commonly urge or even mandate that patients in the plans that they administer obtain their maintenance medications from mail order pharmacies. It is a widely held belief that mail order pharmacies may have lower operating costs and may offer greater discounts available on medication coverage. To the extent that such is the case, use of mail order pharmacies may be a desirable cost control strategy if other terms of agreement remain equalized. Moreover, there is some evidence suggesting that mail order pharmacies promote better patient adherence by commonly providing 90-day supplies and delivering directly to a patient's home (resulting in fewer gaps in a patient's ready access to a prescribed medication). Mail order pharmacies often also receive higher quality and safety ratings than pharmacies. However, to the extent that business is diverted from pharmacies to mail order pharmacies, the former are deprived of income and their ability to survive to provide the local service is impaired. This is true even when a local pharmacy is part of a larger chain pharmacy organization, since decline in income of a local site could lead the chain to close that local site, notwithstanding that other locations of the chain's pharmacies remain in business. There may be advantages to maintaining access to local pharmacies. Numerous studies have established that for many prescription patients, direct contact with a pharmacist is very important. Professional pharmacists are held in very high regard by patients and their advice is eagerly sought. Most patients are not knowledgeable about medications and a prescribing physician's schedule may not provide sufficient time for a patient to be able to get what he or she believes to be sufficient information from the prescribing physician about all aspects of concern about a prescribed medication. Patients want to be able to speak directly to a skilled health care professional for more information about their medications, especially when a long-term maintenance medication is involved. The prospects for a patient's successful implementation of a medication regimen are enhanced when the patient understands and is comfortable with the medication prescribed. Accordingly, there is utility in maintaining local pharmacies as an option for patients, or, in the alternative, providing patients with a user-friendly web-based portal, software application, and/or call center through which they can learn more about their health conditions and prescribed medications.

Because the cost of medications is so high and is such a large component of medical care costs generally, there is an on-going effort on the part of PBMs and payers to seek ways to control medication costs. Pharmaceutical medications are commonly available to patients (patients) either in proprietary (“brand name”) or generic form. In some cases, especially for new or patented medications, only the brand name medication is available, usually only from a single source—the developer of the medication. For many others, however, there is no proprietary limitation on manufacture of the medication, and multiple sources of the medication—in generic or alternative brand name form—are available. Further, it is common that for a given class of pharmaceuticals, there are several different medications with substantially equivalent medical and physiological effects. Some of these medications may be proprietary brand name products while others may be generic and yet others may be available in both generic and brand name forms. prices charged by pharmacies can vary widely, especially where a particular medication is available in both brand name and generic form.

Physicians and other health care providers who write prescriptions therefore often have choices among the different medications they can prescribe for a patient. A physician can, for instance, prescribe a brand name medication or a generic form of that medication, or the physician can choose between two or more different but equivalent medication compositions. There are significant differences in cost among the different medication forms, with generic forms normally being substantially lower in cost than brand name medications. However, even within each group (brand name or generic) there can be substantial cost differences, depending usually on the wholesale prices set by the various manufacturers. Prices can also vary significantly between pharmacies. Currently, however, many patients select the closest pharmacy without comparing prices with mail order options or other pharmacies down the street.

Additionally, it is extremely difficult for patients to compare pharmacies according to other factors such as pharmacy expertise, performance ratings, and/or the appropriateness of substitute or generic medications. As such, most patients are not properly qualified to make assessments of which pharmacy is best for their particular prescription fulfillment. Consequently, needs go unmet and costs are not optimally controlled. Many patients today are unable to optimally select the best pharmacy based on their needs and prescription drug costs.

What would instead be desired is a total fulfillment system, such as the system described herein. In the following description, “prescription” and “Rx” are each intended to mean any prescribed medical good or service. In the following description, references to pharmacies may include and/or mail-order pharmacies, as well as specialty providers. As used herein, specialty providers include specialty pharmacies, such as, for example, dispensers of biologic therapies, vitamins, supplements, and/or over-the-counter medications, and other specialty providers, such as, for example, providers of imaging services or other healthcare services.

In some embodiments of a total healthcare needs fulfillment system, the pharmacy best suited to fulfill a prescription is the pharmacy that is actually selected to fulfill the prescription. In some embodiments, the system matches pharmacies to the prescription requests that they are best suited to fulfill. This matching may be accomplished in a manner that reduces costs. In some embodiments, the matching process does not rely solely upon pricing/costs to determine which pharmacy is best suited to fulfill each prescription, but also considers factors such as location and pharmacy performance. In some embodiments, this system simplifies prescription ordering services and operates with minimum burden on the patients using the system, while still providing patients with choice.

The system of some embodiments interfaces with both mail order and pharmacies. In some embodiments, the system works with dispensers of both brand name and generic medications, and as such, is able to compare cost differences between equivalent medications. For example, in some embodiments, the system may determine if prescription drug substitutions are appropriate, and if so, select a pharmacy that supplies the lowest priced medication, be it generic or brand name, when a physician writes a prescription for a brand name drug. In some embodiments, the system coordinates a bidding process with pharmacies/suppliers to determine which pharmacies supply the lowest priced medication.

In some embodiments, the system provides a central portal for a patient to manage and view all of his/her prescriptions in one place, while the system may also divide and send the patient's prescriptions to different pharmacies based on pharmacies' expertise and prices. Such a system may result in more optimal costs and services and permit pharmacies to more efficiently manage their inventories.

The system of some embodiments also provides a central resource which patients can access to ask questions regarding prescription orders, copays, prescription benefits, and their healthcare, health conditions, and general health.

The total fulfillment system of some embodiments provides an online tracking system allowing patients, doctors, and other users of the system to track prescription orders. Such a system may allow users to track when an order has been received by a pharmacy, adjudicated, fulfilled, and shipped. The data from such a system could ideally be used to track prescription ordering histories and to provide data that can be analyzed to determine how to increase prescription fulfillment efficiencies.

### System Overview

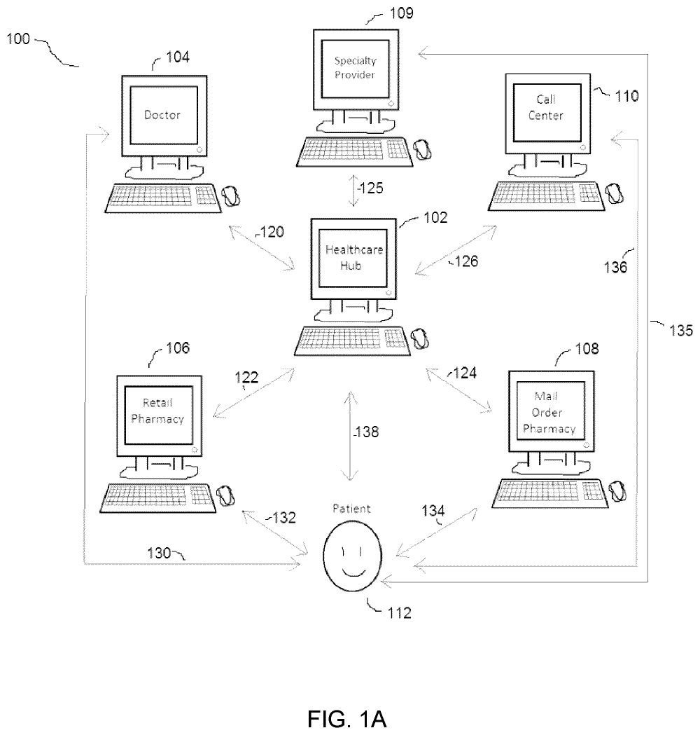

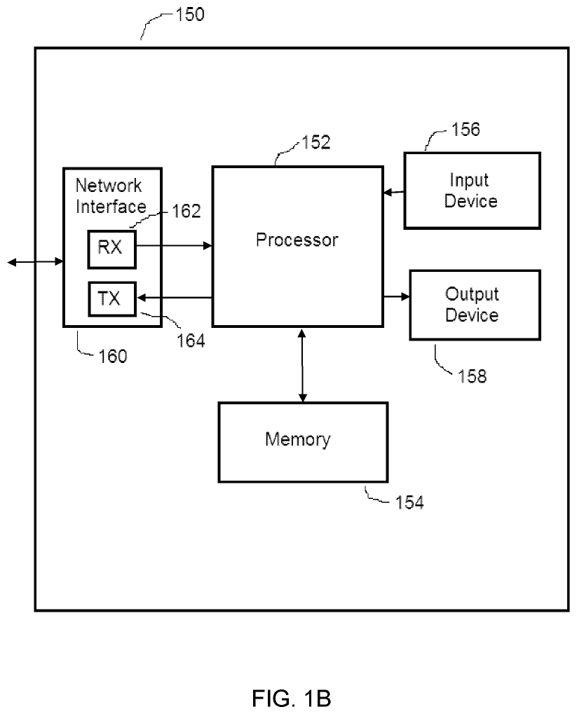

FIG. 1A is a block diagram of one embodiment of a healthcare needs fulfillment system 100. The diagram depicts the system participants and interactions between the participants. The healthcare needs fulfillment system 100 is operated by the healthcare hub 102. The healthcare hub 102 receives information from, and sends information to, doctors 104, pharmacies 106, mail order pharmacies 108, specialty providers 109, call centers 110, and patients 112. (As used herein, “doctor” may refer to a physician, nurse, physician's assistant, or other employee of a physician.) The patient 102 is also able to interact with each participant in the system, and in some embodiments, the patient 102 exchanges information with the patient's doctor 104, a call center 110, a pharmacy 106, a mail order pharmacy 108, a specialty provider 109, and/or the healthcare hub 102.

The healthcare hub 102 is configured to exchange information with the system participants in order to manage prescription fulfillment. The healthcare hub 102 of various embodiments is configured to achieve one or more of the following objectives: reduce healthcare costs, minimize the burden of prescription management experienced by the patient, ensure that the patient receives the correct prescribed goods or services, and provide an avenue for the patient to receive answers to health and healthcare-related questions. In some embodiments, the healthcare hub 102 achieves one or more of the following objectives through the exchange of information with the system participants. The healthcare hub 102 receives information from a doctor 104 via a communication link 120. This information can include, for example, a prescription (script) for a patient 112 and information about the patient 112. The information about the patient 112 can include, for example, a patient-specific identifier and/or information about a patient's health conditions, prescribed drugs, medical history, height, weight, and/or birthdate. The healthcare hub 102 can transmit information back to the doctor 104 via the communication link 120. In some embodiments, the communication link 120 is a two-way (forward and reverse) communication link. The healthcare hub 102 may, for example, request additional information from the doctor 104. In some embodiments, the healthcare hub 102 requests clarification about whether a prescribed brand name drug can be substituted with a comparable generic drug. The healthcare hub 102 may also contact the doctor 104, for example, to inquire about gaps in care, to communicate best practices in care, to provide tips for managing a patient's disease state, or to alert the doctor 104 that the patient 112 is currently receiving prescriptions that may negatively interact with a prescription newly prescribed by the doctor 104. The communication link 120 of some embodiments includes an internet connection. In other embodiments, the communication link 120 may be a telephonic or facsimile connection.

In some embodiments, once the healthcare hub 102 has received a prescription from a doctor 104 and verified that it is an appropriate prescription, the healthcare hub 102 may transmit the prescription to a pharmacy 106, a mail order pharmacy 108, or a specialty provider 109 via communication link 122, communication link 124, or communication link 125, respectively. In some embodiments, the communication links 122, 124, and 125 are two-way (forward and reverse) communication links. The pharmacies 106, mail order pharmacies 108, and specialty providers 109 of some embodiments send notifications to the healthcare hub 102 via communication link 122 as a prescription is processed through the respective pharmacy's system. For example, in some embodiments, the pharmacy 106 will send notifications to the healthcare hub 102 via the communication link 122 when the prescription order has been received, processed, and filled. This may allow the healthcare hub to generate prescription status updates. Similarly, in some embodiments, the mail order pharmacy 108 and specialty providers send notifications to the healthcare hub 102 via the communication links 124 and 125, respectively, when a prescription order has been received, processed, filled, and if applicable, deducted for in a patient's payment account and/or shipped. Again, this may allow the healthcare hub 102 to generate prescription status updates. Additionally or alternatively, notifications sent to the healthcare hub 102 from the pharmacies 106, 108 and specialty providers 109 provide the healthcare hub 102 with data that it can store, analyze, and/or report regarding the speed, error rate, etc. of the respective pharmacies 106, 108 and specialty providers 109. Such data enables the healthcare hub 102 to evaluate and benchmark pharmacy performance.

In other embodiments, the healthcare hub 102 sends requests for bids related to particular medical goods or services to pharmacies 106, mail order pharmacies 108, and/or specialty providers 109 via the respective communication links (122, 124 and/or 125). Additionally or alternatively, in some embodiments, the healthcare hub 102 provides various pharmacies 106, 108 and/or specialty providers 109 with access to a bidding system, such as, for example, by sending an electronic invitation or a link to a bidding website. In response, the pharmacies 106 and/or mail order pharmacies 108 may provide bids back to the healthcare hub 102 via the respective communication links (122, 124 and/or 125). In various embodiments, the bids are submitted electronically to the healthcare hub 102 through a web-based portal.

In some embodiments, the communication links 122, 124, and/or 125 may be telephonic or facsimile connections. In a preferred embodiment, one or more of the communication links 122, 124, and 125 are web-based and/or internet-based connections.

In some embodiments, the healthcare hub 102 applies a rule engine, performs calculations, and/or performs comparisons to determine if a prescription is being filled by the best or lowest-priced pharmacy 106. If the healthcare hub 102 determines that one or more better options exist, the healthcare hub 102 of some embodiments provides information about the one or more better options to a call center 110 via a communication link 126. In some embodiments, the call center 110 is operated by an outside vendor operating under an agreement with the healthcare hub 102. In other embodiments, the call center 110 is a call center facility, an individual employee, or an automated calling system operated by the healthcare hub 102. In other embodiments, the call center 110 may be a facility, company, individual or computer that communicates with patients electronically via text messaging, emailing, or internet-based messaging. The call center 110 of some embodiments initiates a call or other communication with a patient 112 via communication link 136 to determine if the patient 112 would like to switch pharmacies. The call center 110 of some embodiments also provides the patient 112 with additional healthcare tips, prescription reminders, healthcare adherence reminders, and/or other healthcare-related content after receiving such tips, reminders, and content from the healthcare hub 102 via the communication link 126. In some embodiments, the communication link 126 is a two-way (forward and reverse) communication link. For example, in some embodiments, the call center 110 transmits authorization information via communication link 126 back to the healthcare hub 102, indicating whether the patient authorized a pharmacy switch.

In some embodiments, the patient 112 can send patient-specific information, prescriptions received from a doctor 104, and/or healthcare questions to the healthcare hub 102 via communication link 138. The healthcare hub 102 of some embodiments sends prescription status updates to the patient 112 via the same communication link 138. In some embodiments, the healthcare hub 102 provides a web-based interface through which a patient 112 can easily view all of the patient's prescribed medical goods and services—even if several pharmacies and suppliers are providing the various goods and services. In some such embodiments, a patient logged into a web portal can track the status of each prescribed good and service and provide financial information to facilitate the automatic payment of copays. Additionally or alternatively, in some embodiments, the healthcare hub 102 is configured to provide answers to the patient's questions, feedback from a care team, and/or health education content via communication link 138. In some embodiments, the healthcare hub 102 will alert a patient 112 of potential drug interactions, remind a patient to adhere to a healthcare or wellness regimen, alert a patient 112 when healthcare goods or services utilized by the patient 112 are available at a “better pharmacy”, and/or provide a patient 112 with educational health and wellness tips. The healthcare hub 102 of some embodiments provides personalized content related to disease states and/or health topics of relevance to a particular patient 112 via communication link 138. In some such embodiments, the content is delivered to the patient in a highly accessible format to empower the patient to make informed health, wellness, and healthcare decisions. For example, in some embodiments, communication link 138 is a wireless communication link, and content is sent from the healthcare hub 102 to the patient 112 via text messaging or email or through a web-based portal or software application. In some embodiments, the healthcare hub 102 may connect the patient 112 directly to a doctor 104, pharmacy 106, mail order pharmacy 108, specialty provider 109, or call center 110 in order to have the appropriate party answer the patient's questions. In some such embodiments, the patient 112 exchanges information back and forth with the doctor 104, the pharmacy 106, the mail order pharmacy 108, the specialty provider 109, and/or the call center 110 via communication link 130, 132, 134, 135 and 136, respectively.

In some embodiments, the doctor 104, the pharmacy 106, the mail order pharmacy 108, the specialty provider 109, the call center 110, and the patient 112 are all connected to the healthcare hub 102 via a network, for example, the Internet. In some embodiments, the patient 112 is also connected to the doctor 104, the pharmacy 106, the mail order pharmacy 108, the specialty provider 109, and the call center 110 via a network, such as the Internet. In other embodiments, the patient 112 exchanges information with the doctor 104, the pharmacy 106, the mail order pharmacy 108, the specialty provider 19, and/or the call center 110 via a telephonic connection or in-person interaction. Thus, it is to be appreciated that, in at least some embodiments, the communication links 120, 122, 124, 125, 126, 130, 132, 134, 135, 136, and 138 are representative of the communication functionality rather than physical connections.

The healthcare hub 102 of some embodiments is a computing system (herein referred to as a healthcare needs fulfillment system), which includes, at least, a processor in data communication with a memory and a network interface. FIG. 1B provides a functional block diagram of one embodiment of such a healthcare needs fulfillment system 150. Although described separately, it is to be appreciated that functional blocks described with respect to the healthcare needs fulfillment system 150 need not be separate structural elements. For example, the processor 152 and memory 154 may be embodied in a single chip. Similarly, the processor 152 and network interface 160 may be embodied in a single chip. Likewise, the receiver 162 and transmitter 164 may be embodied in a single chip.

The processor 152 can be a general purpose processor, a digital signal processor (DSP), an application specific integrated circuit (ASIC), a field programmable gate array (FPGA) or other programmable logic device, discrete gate or transistor logic, discrete hardware components, or any suitable combination thereof designed to perform the functions described herein. A processor may also be implemented as a combination of computing devices, e.g., a combination of a DSP and a microprocessor, a plurality of microprocessors, one or more microprocessors in conjunction with a DSP core, or any other such configuration.

The processor 152 is coupled, via one or more buses, to read information from or write information to the memory 154. The processor may additionally, or in the alternative, contain memory, such as processor registers. The memory 154 can include processor cache, including a multi-level hierarchical cache in which different levels have different capacities and access speeds. The memory 154 can also include random access memory (RAM), other volatile storage devices, or non-volatile storage devices. The storage devices can include hard drives, optical discs, such as compact discs (CDs) or digital video discs (DVDs), flash memory, floppy discs, magnetic tape, and Zip drives.

The processor 152, in conjunction with software stored in the memory 154 executes an operating system, such as, for example, Windows, Mac OS, Unix or Solaris 5.10. The processor 152 also executes software applications stored in the memory 154. For example, the functionality for identifying one or more suggested pharmacies for a patient can be programmed as software stored in the memory 154. In one non-limiting embodiment, the software comprises, for example, Unix Korn shell scripts. In other embodiments, the software can be programs in any suitable programming language known to those skilled in the art, including, for example, C++ and Java.

In one embodiment, the memory 154 may include software for operating the healthcare needs fulfillment system 150 as a web server, such as for example, the software provided by Apache and Tomcat. In one embodiment, the memory 154 includes a web-accessible database, that is, a database which is accessible via the network interface 160. Software stored in the memory 154, such as for example, Oracle 10g, provides database services to the processor 152 and to users of the healthcare needs fulfillment system 150.

The processor 152 is also coupled to an input device 156 and an output device 158 for, respectively, receiving input from and providing output to, a system administrator of the healthcare needs fulfillment system 150. Suitable input devices include, but are not limited to, a keyboard, buttons, keys, switches, a pointing device, a mouse, a joystick, a remote control, an infrared detector, a video camera (possibly coupled with video processing software to, e.g., detect hand gestures or facial gestures), a motion detector, and a microphone (possibly coupled to audio processing software to, e.g., detect voice commands). Suitable output devices include, but are not limited to, visual output devices, including displays and printers, audio output devices, including speakers, headphones, earphones, and alarms, and haptic output devices, including force-feedback game controllers and vibrating devices.

The processor 152 may be further coupled to a network interface 160, including a receiver 162 and a transmitter 164. The transmitter 164, in conjunction with the network interface 160, prepares data generated by the processor 152 for transmission over a communication network according to one or more network standards. The receiver 162, in conjunction with the network interface 160, demodulates data received over a communication network 125 according to one or more network standards. In one embodiment, the transmitter 164 and the receiver 162 are part of the same component, such as, for example, a transceiver. In other embodiments, the transmitter 164 and receiver 162 are two separate components.

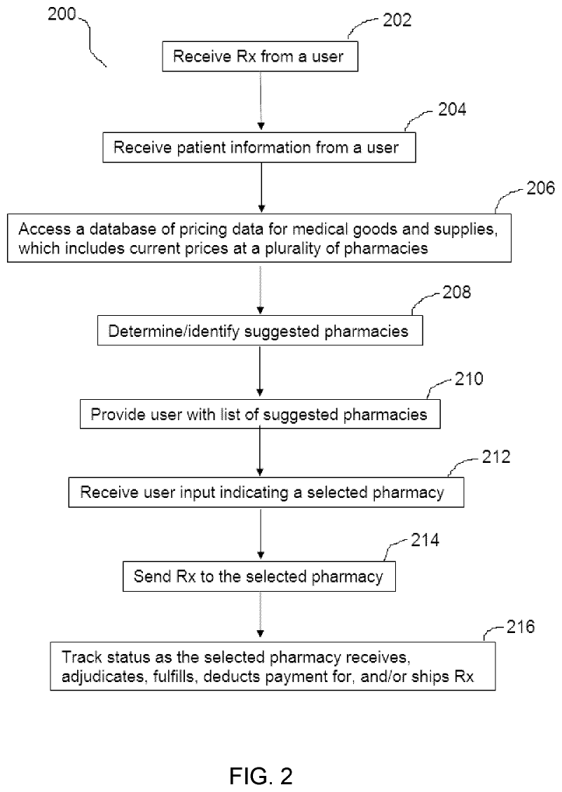

FIG. 2 provides a flowchart illustrating one embodiment of a method 200 for fulfilling and managing prescriptions performed by the healthcare needs fulfillment system 150 of FIG. 1B. In the illustrated embodiment, the healthcare needs fulfillment system receives a prescription from a user, as shown in block 202. As used herein, a user may be a patient, a physician, or an employee or contractor of a healthcare facility. The prescription includes, at least, the name of a prescription drug or medical good or service and, if applicable, a dosage and/or prescribed frequency of use, as prescribed by a healthcare professional for a patient. In some embodiments, the user submits an electronic prescription to the healthcare needs fulfillment system by logging into a web-based interface and entering the electronic prescription into appropriate windows or boxes within the web-based interface. The electronic prescription is transmitted to the healthcare needs fulfillment system over a network connection. In other embodiments, the prescription may be emailed, faxed, or called in to an operator of the healthcare hub. A human or computer affiliated with the healthcare hub can then input the prescription information into the healthcare needs fulfillment system.

In some embodiments, the healthcare needs fulfillment system also receives patient information from a user, as shown in block 204. The patient information may also be entered electronically into a web-based interface and transmitted to the healthcare needs fulfillment system via a network. Alternatively, the patient information may be called in, faxed, or emailed to the healthcare hub along with the prescription. The patient information of some embodiments includes, at least, an identifier linking the patient to the patient's respective insurance and/or prescription drug plan. In some embodiments, the identifier simply comprises the patient's name and the group number or member code of the patient's insurance and/or prescription drug plan.

In other embodiments, the identifier is a username or access ID, which uniquely identifies the patient and links the patient to a patient-specific data space within the memory of the healthcare needs fulfillment system. In such embodiments, the patient-specific data space stores data pertaining to a patient-specific profile. The patient-specific profile of some embodiments is maintained by the healthcare needs fulfillment system and accessible by the patient via the web-based interface. In some embodiments, the patient-specific profile is editable by the patient and/or the patient's physician. The patient-specific profile of some embodiments includes patient-specific information, including at least the patient's: prescription benefit plan, birthdate, name, address, and current prescriptions. In some embodiments, the patient-specific profile may also include the patient's medical history or other biographical information. In some embodiments in which a patient-specific profile is present, the healthcare needs fulfillment system creates or updates a patient-specific profile upon receiving patient information from a user.

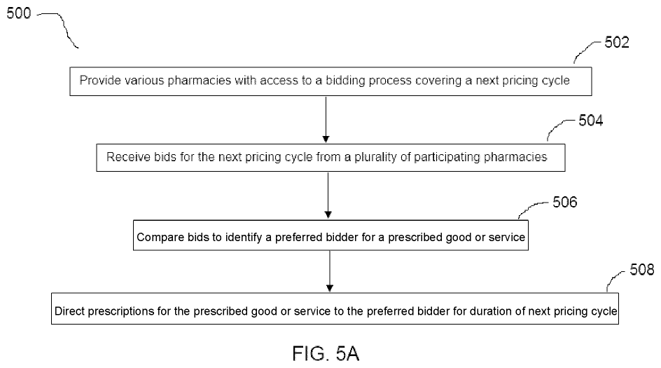

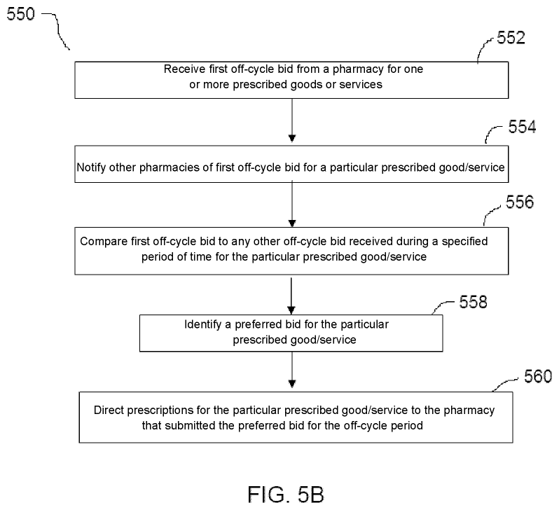

Returning to FIG. 2, after receiving a prescription and patient information from a user, the healthcare needs fulfillment system accesses a database of pricing data for medical goods and services, as indicated by block 206. In some embodiments, the database of pricing data is stored remotely from the healthcare needs fulfillment system and is accessed via a network, such as the Internet. In other embodiments, the database of pricing data is stored within the memory of the healthcare needs fulfillment system. In some embodiments, the database includes listings of current drug prices, listed by drug and by pharmacy. In some embodiments, the database includes listings of current prices for various medical goods and services, listed, for example, by manufacturer, trade name, and pharmacy. These prices reflect the prices at which each listed pharmacy has agreed to sell each listed drug or other medical good or service. In some embodiments, the current prices are updated whenever a pharmacy submits a revised list of prices. In other embodiments, the current prices are updated periodically upon receiving bid sheets from a plurality of bidding pharmacies. Such a bidding process is described in more detail below with reference to FIGS. 5A and 5B.

As depicted in block 208, in some embodiments, the healthcare needs fulfillment system identifies one or more suggested pharmacies by evaluating all pharmacies listed within the database based, at least in part, on: (1) the prescription drug name, and if applicable, the prescribed dosage, prescribed amount, and/or prescribed frequency of use, as identified in the prescription, (2) the patient's prescription benefit plan identified from the patient information, and (3) the current pricing data provided in the database of pricing data for medical goods and services. From this information, the healthcare needs fulfillment system of some embodiments can identify one or more pharmacies that will fill the patient's prescription most cheaply.

In other embodiments, the one or more suggested pharmacies may be identified taking into account more than just price. For example, in some embodiments, the healthcare needs fulfillment system also takes into account the address of each pharmacy listed within the database and the address of the patient. The pharmacy addresses may be stored in a database of pharmacy information within the memory of the healthcare needs fulfillment system. The patient's address may be stored within the patient-specific profile. In such embodiments, the one or more suggested pharmacies identified by the system may include, for example, the three pharmacies that will fill the prescription most cheaply within a 3-mile radius of the patient. In other embodiments, the system may be configured to identify a different number of suggested pharmacies and/or use a different distance/radius in its calculations. In some embodiments, the healthcare needs fulfillment system additionally takes into account other data stored within the database of pharmacy information when identifying one or more suggested pharmacies. For example, in some embodiments, the healthcare needs fulfillment system identifies suggested pharmacies based, at least in part, on the expertise and/or quality rating of each pharmacy. In some embodiments, the healthcare needs fulfillment system takes into account all of a patient's prescriptions, as a bundled package, when evaluating pharmacies based on criteria such as price, location, availability, quality, and expertise. In this manner, the system may identify one or more suggested pharmacies best aligned for managing all of a patient's prescribed healthcare needs.

In some embodiments, the healthcare needs fulfillment system provides the user with a list of suggested pharmacies, as shown in block 210, and receives a user input, as shown in block 212, indicating which suggested pharmacy the user has selected to be the patient's new pharmacy. The list may be transmitted to the user over the Internet and displayed to the user via the web-based interface. Similarly, using the web-based interface, the user can provide an input to select the new pharmacy. In some embodiments, only pharmacies and/or specialty providers are shown in the list of suggested pharmacies. In other embodiments, mail-order is included as one of the user's options in the list of suggested pharmacies. Upon receiving the user's input identifying the suggested pharmacy selected to be the patient's new pharmacy, the prescription is sent in electronic form to the selected new pharmacy, as shown in block 214.

In some embodiments of a method for fulfilling and managing prescriptions performed by a healthcare needs fulfillment system, the operations disclosed in blocks 210 and 212 are not included. In such embodiments, after receiving a prescription and patient information from a user and accessing a database of pricing data for prescription drugs and/or other medical goods, the healthcare needs fulfillment system identifies one suggested pharmacy at block 208. As described above, the suggested pharmacy determination may be based on a pharmacy's price for a particular good or service, proximity to the patient, particular expertise, speed of fulfillment, availability of the good or service, quality rating, overall price or quality for a bundle of prescriptions, and/or other factors. Once the suggested pharmacy is identified, the prescription is sent electronically to that pharmacy.

In some embodiments, the method for fulfilling and managing prescriptions further includes tracking the status of the prescription through the pipeline. That is, in some embodiments, the status of the prescription can be tracked electronically as the selected pharmacy receives, adjudicates, fulfills, bills for, and/or ships the prescription. Such an operation is shown at block 216. If the selected pharmacy is a mail order pharmacy, the healthcare needs fulfillment system may additionally be able to track the status of the shipping process from drop off to delivery. In some embodiments, the selected pharmacy provides notifications to the healthcare needs fulfillment system when the status of the prescription has changed, thereby allowing the healthcare needs fulfillment system to track the status of the prescription. In some embodiments, the status of the prescription is viewable by the user through the web-based interface.

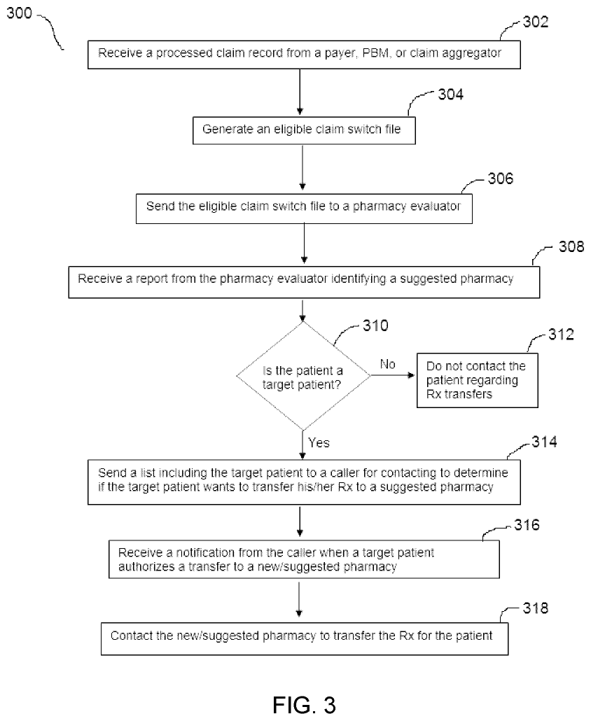

FIG. 3 provides a flowchart illustrating another embodiment of a method for fulfilling and managing prescriptions. In the depicted method 300, the healthcare needs fulfillment system receives a processed claim record from a payer, PBM, or claim aggregator at block 302. The processed claim record includes information about at least one prescription fulfilled at a pharmacy. The processed claim record at least identifies the fulfilling pharmacy, the patient, and the prescribed good or service for each fulfilled prescription. From this information, and optionally, from additional patient information stored in a patient-specific profile, the healthcare needs fulfillment system generates an eligible claim switch file as shown at block 304. The eligible claim switch file identifies one or more prescriptions that may be good candidates for transfer to another pharmacy.

At block 306, the eligible claim switch file is sent to a pharmacy evaluator. The pharmacy evaluator of some embodiments is an outside vendor. In some embodiments, the eligible claim switch file is sent to the pharmacy evaluator via the web-based interface. In other embodiments, the pharmacy evaluator is a sub-system of the healthcare needs fulfillment system, and the eligible claim switch file is sent to the pharmacy evaluator via a LAN, intranet, or electronic connection. The pharmacy evaluator generates a report identifying one or more suggested pharmacies. The one or more suggested pharmacies are pharmacies that would fulfill the prescription most optimally, as determined by taking into account one or more factors. Such factors may include, for example, a pharmacy's pricing, proximity to the patient, particular expertise, speed of fulfillment, availability of the good or service, quality rating, and/or performance rating. At block 308, the healthcare needs fulfillment system receives the report from the pharmacy evaluator identifying one or more suggested pharmacies.

At block 310, the healthcare needs fulfillment system evaluates whether the patient is a target patient. In various embodiments, the patient is found to be a target patient if the fulfilling pharmacy listed in the processed claim record is not listed within the report of suggested pharmacies. Such a finding indicates that the patient's prescription is not currently being filled optimally. For example, such a finding may suggest that one or more pharmacies are closer to the patient, cheaper for the patient, have more expertise with a particular health condition of the patient, and/or are better able to manage a patient's entire bundle of prescriptions than the patient's current pharmacy. In some embodiments, additional considerations are taken into account when determining whether a patient qualifies as a target patient. For example, in some embodiments, the healthcare needs fulfillment system determines whether the patient is permitted to transfer prescriptions under the patient's prescription benefit plan. In one example, the healthcare needs fulfillment system excludes all Medicare Part D recipients from the target patient population.

As shown at block 312, if the patient is not identified as a target patient, the patient will not be contacted to discuss a prescription transfer and the patient's prescriptions will not be transferred. If the patient is identified as a target patient, as in block 314, the patient will be included within a list of target patients that the healthcare needs fulfillment system sends to a caller. The list will include, at least, the patient's name, the patient's contact information, the patient's prescription, and the one or more suggested pharmacies. In some embodiments, the caller is an outside vendor contracted to make calls for the healthcare needs fulfillment system. In some such embodiments, the list of target patients is provided to the caller via a web-based interface. In other embodiments, the caller is an automated calling sub-system, which forms part of the healthcare needs fulfillment system. The list of target patients may be provided to the sub-system, for example, via a LAN, intranet, or electronic connection. In some embodiments, the caller contacts each target patient included on the list to determine if each target patient would like to transfer the patient's respective prescription to one of the suggested pharmacies. The caller may contact each target patient via phone, email, or mail. In some embodiments, the caller provides the target patient with the location and/or the required copay associated with each of the suggested pharmacies to better inform the patient's decision.

In some embodiments of the method, the healthcare needs fulfillment system receives a notification from the caller when a target patient authorizes a transfer of the patient's prescription to one of the suggested pharmacies, as shown at block 316. The healthcare needs fulfillment system contacts the pharmacy selected by the target patient and transfers the prescription for the patient, as shown at block 318. Conversely, in other embodiments, when a target patient in contact with the caller authorizes a prescription transfer, the target patient is instructed to bring the prescription to the selected pharmacy. In other embodiments, when a target patient in contact with the caller authorizes a prescription transfer, the caller contacts the selected pharmacy and transfers the prescription for the patient.

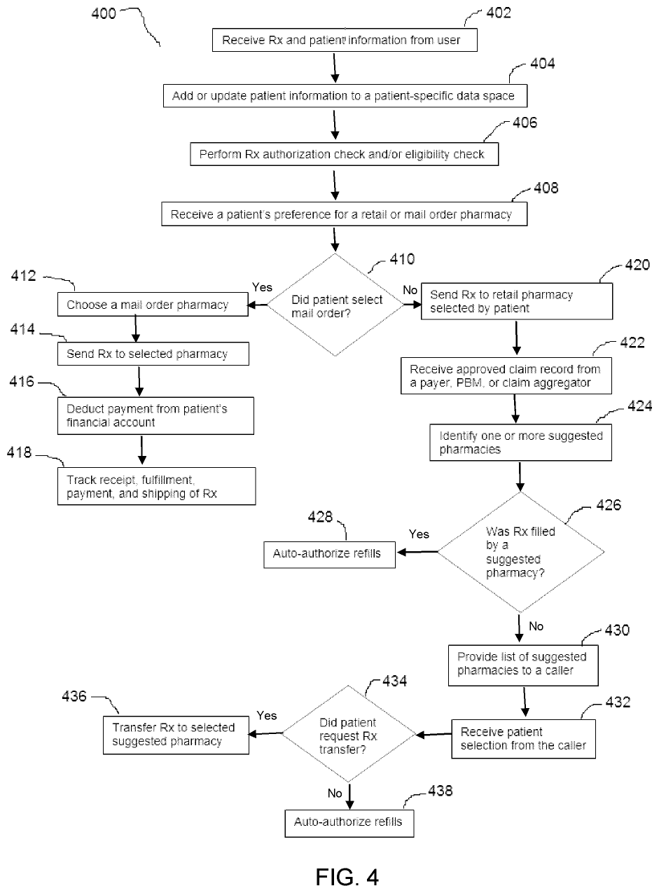

FIG. 4 is a flowchart illustrating another embodiment of a method for fulfilling and managing prescriptions. In the method 400 of FIG. 4, the healthcare needs fulfillment system receives prescription and patient information from a user, as described above in the description of FIG. 2. Upon receiving the information at block 402, the healthcare needs fulfillment system updates a patient-specific profile to include at least some of the information received at block 402. As shown at block 404, if a patient-specific profile does not yet exist for a particular patient, the healthcare needs fulfillment system of some embodiments will generate a patient-specific profile upon receiving patient information from the user.

At block 406, the healthcare needs fulfillment system performs a prescription authorization and/or eligibility check. In some embodiments, the authorization check includes comparing the received prescription to the patient information to determine, for example, whether the prescribed good or service, and dosage or frequency of use, if applicable, is consistent with the patient's diagnosis and/or whether the prescribed good or service might cause adverse effects with other prescriptions of the patient. In some embodiments, the eligibility check includes verifying the patient's insurance or prescription benefits plan and/or determining whether the prescribed good or service is covered by the patient's plan. Additionally or alternatively, in some embodiments, where applicable, the authorization check includes contacting the patient's physician to determine whether a generic drug is an acceptable substitute for a brand name drug. The prescription authorization check of some embodiments is fully automated. In other embodiments, at least a portion of the prescription authorization check is performed by a healthcare needs fulfillment system administrator.

At optional block 408, the healthcare needs fulfillment system receives a patient's preference for mail-order or a particular pharmacy. Upon receiving the preference, the healthcare needs fulfillment system processes the preference at block 410.

If the patient selected to receive the prescription via mail order, or if optional block 408 is not present in the system, the healthcare needs fulfillment system choses a mail-order pharmacy to be the provider of the prescription, as shown at block 412. The chosen mail-order pharmacy may be selected at least in part via a bidding system, as described in detail below with reference to FIGS. 5A and 5B. Additionally, or in the alternative, the chosen mail-order pharmacy may be selected through an evaluation process which weighs one or more criteria such as, for example, the prescription sales prices, quality ratings, safety ratings, and expertise of various pharmacies. At block 414, the healthcare needs fulfillment system sends the prescription to the selected mail-order pharmacy.

Optionally, in some embodiments, the healthcare needs fulfillment system deducts payment for the prescription copay from the patient's bank account (or charges the payment to the patient's credit card) as shown at block 416. To make the operation shown at block 416 possible, a patient's financial account information must be stored in the patient-specific profile. In some embodiments, a patient can add or modify financial account information and/or other patient information included in the patient-specific profile at any time via the web-based interface.

In some embodiments, the healthcare needs fulfillment system tracks the receipt, fulfillment, payment, and/or shipment of the prescription by the mail order pharmacy, as shown at block 418. The healthcare needs fulfillment system of such embodiments provides prescription status updates, which the patient or other user of the system can access.

If, at block 410, the healthcare needs fulfillment system determined that the patient selected a particular pharmacy, the method proceeds to block 420, and the prescription is sent to the pharmacy selected by the patient for fulfillment. Upon receiving an approved claim record from a payer, PBM, or claim aggregator (see block 422), a method similar to the method embodied in FIG. 3 is performed. At blocks 424 and 426, the healthcare needs fulfillment system determines whether the prescription was filled by the best pharmacy for the patient. This determination is performed by identifying one or more “best” or suggested pharmacies, based on factors such as price, proximity to the patient, availability of the prescribed good or service, speed of fulfillment, pharmacy performance, and/or pharmacy expertise and comparing the patient-selected pharmacy to the identified best pharmacies. As described previously, in some embodiments, an outside vendor identifies one or more suggested pharmacies for filling the patient's prescription. The healthcare needs fulfillment system then determines whether the prescription was filled by the best pharmacy for the patient by evaluating whether the patient-selected pharmacy is included in the vendor's list of one or more suggested pharmacies.

In some embodiments, if it is determined that the prescription was filled by one of the best or suggested pharmacies, the healthcare needs fulfillment system will auto-authorize refills of the prescription by the current pharmacy in the future (as shown at block 428). If, on the other hand, it is determined that the prescription was not filled by one of the best or suggested pharmacies, the method proceeds to block 430. In some embodiments, a list of suggested or better pharmacies is provided to a caller. As before, the caller may be a person or a computerized system, and the caller may be an outside vendor or a subsystem of the healthcare needs fulfillment system. In some embodiments, after the caller contacts the patient (via phone, fax, email, text, etc.) and receives a response from the patient, the response is forwarded to the healthcare needs fulfillment system. At block 432, the system receives the patient's response from the caller. At block 434, the healthcare needs fulfillment system analyzes the patient's response to determine whether the patient requested a prescription transfer to a new pharmacy. As shown at block 436, if the patient requested a transfer, the healthcare needs fulfillment system of some embodiments transfers the prescription to the new pharmacy. In some embodiments, if the patient does not request a transfer, the healthcare needs fulfillment system will auto-authorize refills of the prescription by the current pharmacy in the future (as shown at block 438).

FIG. 5A is a flowchart illustrating an embodiment of a method performed by a healthcare needs fulfillment system for identifying a preferred provider of a prescription. In the illustrated embodiment of the method 500, the healthcare needs fulfillment system employs a bidding process to help identify the preferred providers. For example, in block 502, the healthcare needs fulfillment system provides various pharmacies with access to the bidding process covering a next pricing cycle. In some embodiments, the prices are set quarterly, so the next pricing cycle is equivalent to the next fiscal quarter. In other embodiments, other pricing cycles are used, such as, for example, daily, weekly, monthly or annually.

At block 504, the healthcare needs fulfillment system receives completed bids for the next pricing cycle from a plurality of participating pharmacies. Each completed bid includes, at least, the price the respective pharmacy will charge for each prescribed good or service. In some embodiments, the completed bids additionally include the price the pharmacy will charge for each formulation and dosage, if applicable, and/or price variations the pharmacy will charge based on a patient's pharmacy benefits plan. In some embodiments, bids are submitted on a per unit basis. For example, in some embodiments, all bids for medical goods and services are listed as: per tablet, per capsule, per test strip, per MRI, etc. In some such embodiments, the per unit basis is the only price the healthcare needs fulfillment system will consider. Accordingly, in such embodiments, each bidder must total all costs they wish to be reimbursed for, such as, for example, the wholesale cost of the good or service, shipping costs, dispensing fees, etc., then perform division to determine the cost per unit. By requiring all bids to be submitted on a per unit basis, the healthcare needs fulfillment system is able to easily and efficiently compare costs across pharmacies.

At block 506, the healthcare needs fulfillment system compares the completed bids to identify the preferred bidder for each good or service, and in some embodiments, for each formulation, each dosage or size, and/or each pharmacy benefits plan. As in other embodiments, the preferred bidder may be selected based on various factors, such as, for example, a pharmacy's prices, fulfillment speed, quality, error rates, and expertise. Information related to one or more of the above-listed factors or other factors may need to be provided by pharmacies during the bidding process. In some embodiments, the bidding is an open process, allowing the participating pharmacies to view the bids of their competitors. In other embodiments, the bidding is closed such that pharmacies cannot view the bids of their competitors. At block 508, the healthcare needs fulfillment system directs prescriptions to the preferred bidder for the duration of the next pricing cycle.

FIG. 5B is a flowchart illustrating another embodiment of a method for identifying a preferred provider of a prescribed good or service through a bidding process. The method 550 of FIG. 5B may be performed by a healthcare needs fulfillment system independently or in conjunction with the bidding process depicted in FIG. 5A. The bidding process of FIG. 5B enables pharmacies to offer deals for a period of time that is shorter than the regular pricing cycle. For example, the pharmacies may offer “deals of the day” or “deals of the week.” In the embodiment of FIG. 5B, the healthcare needs fulfillment system receives a first off-cycle bid from a pharmacy for one or more medical goods or services, as shown at block 552. The bid may apply to all formulations, dosages, sizes, and pharmacy benefits plans, or the bid may specify the limitations of the bid. At block 554, the healthcare needs fulfillment system notifies other pharmacies of the first off-cycle bid received. The notice may be sent directly to the pharmacies, such as for example, by mail, phone, fax, or email, or the notice may be posted on the web-based interface. In some embodiments, the other pharmacies are provided with a window of time within which they can submit counter-bids. At block 556, the healthcare needs fulfillment system compares the first off-cycle bid to any other bids received during the specified period of time. At block 558, the healthcare needs fulfillment system identifies the preferred off-cycle bid. The preferred bid may be selected based on lowest price alone or on a plurality of factors, such as the factors discussed above. For the specified off-cycle period of time, prescriptions will be directed to the pharmacy that submitted the preferred bid for a particular good or service, as in block 560.

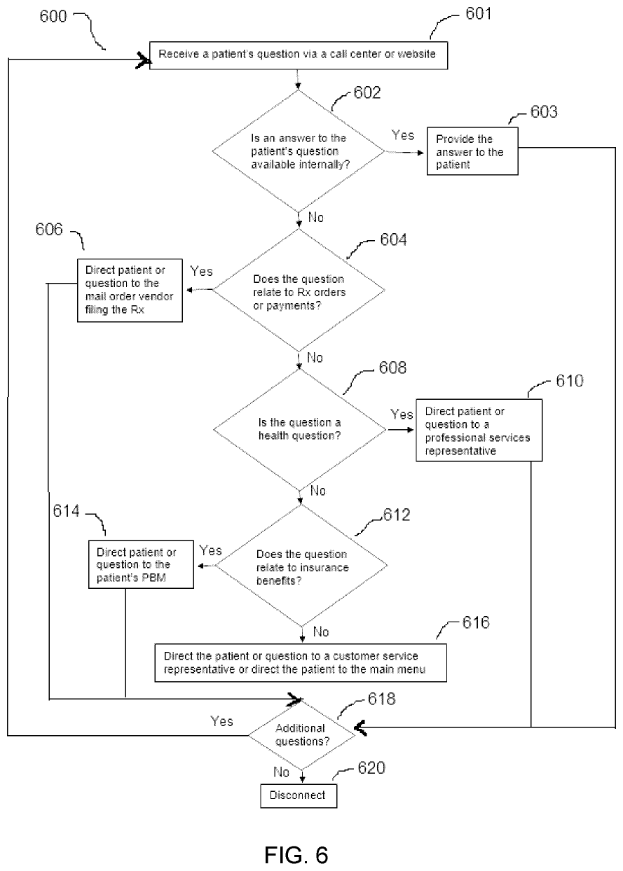

FIG. 6 is a flowchart illustrating an embodiment of a method for answering patients' questions. The method 600 of various embodiments is performed by a healthcare needs fulfillment system. The method 600 can be combined with and/or performed in concert with any of the methods for fulfilling and managing prescriptions described above. As shown at block 601, the healthcare needs fulfillment system of some embodiments receives patient questions through the web-based interface or via a call center. In some embodiments, the operator of the healthcare needs fulfillment system may have the capability of answering some questions directly. In such embodiments, optional block 602 may be performed. At block 602, the system determines whether an answer to a patient's question is readily available onsite. If so, it is answered at block 603. If no, or if optional block 602 is skipped, the patient's question is triaged and the general topic determined in order to direct the question to the most applicable provider of information. In some embodiments, the healthcare needs fulfillment system determines whether the question: (1) relates to prescription orders or payments, as in block 604, (2) relates to patient health topics, as in block 608, or (3) relates to insurance benefits, as in block 612. In some embodiments in which the question is received via a call center, the determination is made by providing relevant automated menu options to the patient and processing the patient's input. For example, in one embodiment, a patient is instructed to “select 1 for questions related to prescription orders or copayments, 2 for questions related to health and wellness, and 3 for questions related to your insurance benefits.” Many other prompts and menu options may be used in other embodiments. In other embodiments, the determination may be made by an operator speaking to the patient. In still other embodiments, the patient submits the question via a web-based interface and is required to specify or select the topic to which the question pertains before submitting the question.

In FIG. 6, if it is determined that the patient's question relates to prescription orders or copayments, the patient or question is directed to the pharmacy that is filling the prescription, as in block 606. If the patient's question relates to health or wellness, the patient or question is directed to a professional services representative, as in block 610. Such representatives may be pharmacists, nurse practitioners, or other healthcare professional qualified to answer the patient's question. If the patient's question relates to insurance benefits, the healthcare needs fulfillment system directs the patient or question to the patient's prescription benefits manager, as in block 614. In some embodiments, if the patient's question does not relate to any of these topics, or if the patient fails to select one of the topics, the patient or question is directed to a customer services representative; in other embodiments, the patient is directed back to a main menu or interface (see block 616). At block 618, the healthcare needs fulfillment system determines whether the patient has one or more additional questions. If the patient has additional questions, the process of triaging and answering the question is repeated; if not, the patient is disconnected (see block 620) or returned to a homepage.

The healthcare needs fulfillment system of various embodiments described herein is intended to manage and coordinate the fulfillment of healthcare needs for thousands or even millions of patients. The system is configured to facilitate communications between hundreds, thousands, or millions of patients, pharmacies, providers, and physicians at any given time.

Those of skill would further appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure.

The various illustrative logical blocks, modules, and circuits described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

In one or more example embodiments, the functions described may be implemented in hardware, software, or firmware executed on a processor, or any combination thereof. If implemented in software, the functions may be stored on or transmitted over as one or more instructions or code on a computer-readable medium. Computer-readable media includes both computer storage media and communication media including any medium that facilitates transfer of a computer program from one place to another. A storage media may be any available media that can be accessed by a computer. By way of example, and not limitation, such computer-readable media can comprise RAM, ROM, EEPROM, CD-ROM or other optical disk storage, magnetic disk storage or other magnetic storage devices, or any other medium that can be used to carry or store desired program code in the form of instructions or data structures and that can be accessed by a computer. Also, any connection is properly termed a computer-readable medium. For example, if the software is transmitted from a web site, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, includes compact disc (CD), laser disc, optical disc, digital versatile disc (DVD), floppy disk and Blu-ray disc where disks usually reproduce data magnetically, while discs reproduce data optically with lasers. Combinations of the above should also be included within the scope of computer-readable media.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes both the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

While the above description has pointed out novel features of the invention as applied to various embodiments, the skilled person will understand that various omissions, substitutions, and changes in the form and details of the device or process illustrated may be made without departing from the scope of the invention. Therefore, the scope of the invention is defined by the claims that follow rather than by the foregoing description. All variations coming within the meaning and range of equivalency of the claims are embraced within their scope.

1. A system for routing electronic prescriptions, the system comprising:
a network interface that includes a receiver and a transmitter, the receiver configured to receive an electronic prescription and patient information over a communications network and the transmitter configured to prepare data for transmission over the communications network;
at least one processor communicatively coupled to the network interface, the at least one processor configured for:
receiving, using the at least one processor and via the communications network, the electronic prescription comprising a prescribed healthcare good associated with a claim for adjudication by a prescription benefits manager (PBM);
receiving, using the at least one processor and via the communications network, the patient information electronically from a patient-specific data store communicatively coupled to the communications network, the patient information comprising an identifier linking a patient to a prescription benefit plan and a fulfilling computing system associated with a fulfilling pharmacy, the patient-specific data store configured to store data pertaining to a patient-specific profile;
accessing, using the at least one processor and via the communications network, a database of pricing data communicatively coupled to the communications network, the database of pricing data stored remotely and accessible via the communications network, the database of pricing data comprising prices of goods at a plurality of participating pharmacies;
selecting, using the at least one processor, a suggested pharmacy accessible to the patient by evaluating the plurality of participating pharmacies at least in part on proximity to the patient and the prices of goods;
sending, using the at least one processor and via the communications network, the prescription electronically to a computing system of the suggested pharmacy for fulfillment;
determining, using the at least one processor, whether a patient question received from the patient via a user interface is to be directed to at least one of the suggested pharmacy, a professional health services representative, or an insurance benefits manager;
routing, using the network interface having the transmitter and in response to determining the patient question received from the patient via the user interface is to be directed to the insurance benefits manager via the communications network, the patient question electronically to the computing system of the insurance benefits manager;
routing, using the network interface having the transmitter and in response to determining the patient question received from the patient via the user interface is to be directed to the professional health services representative via the communications network, the patient question electronically to the computing system of the professional health services representative; and
routing, using the network interface having the transmitter and in response to determining the patient question received from the patient via the user interface is to be directed to the suggested pharmacy via the network interface, the patient question electronically to the computing system of the suggested pharmacy.

2. The system of claim 1, wherein the computing system of the suggested pharmacy is automatically chosen to be a pharmacy associated with the patient.

3. The system of claim 1, wherein identifying the suggested pharmacy comprises:
identifying, via the communications network, a plurality of computing systems associated with suggested pharmacy, the plurality of computing systems communicatively coupled to the communications network,
providing, via the communications network, a list of the computing systems associated with the suggested pharmacy to a user in a selectable format, and
receiving, via the communications network, an input from the user indicating a selected computing system of a selected pharmacy, the selected computing system of the selected pharmacy communicatively coupled to the communications network.

4. The system of claim 1, wherein the plurality of participating pharmacies participate in a bidding process that includes a first off-cycle bid and a second off-cycle bid.

5. The system of claim 4, wherein the first off-cycle bid and the second off-cycle bid are valid for a second period of time that is shorter than a first period of time associated with a regular pricing cycle.

6. The system of claim 1, wherein the identifier is a patient-specific identification code or username linking the patient to the patient-specific profile, which stores patient-specific information, including at least one of the prescription benefit plan, a birthdate, a name, and an address.

7. The system of claim 1, wherein the patient-specific profile also stores financial account information for the patient, and wherein the at least one processor further comprises deducting payment from the financial account information when the electronic prescription is received, adjudicated, or fulfilled by a selected computing system of a selected pharmacy.

8. The system of claim 1, further comprising:
generating, using the at least one processor, a claim switch for authorization, in response to determining that the suggested pharmacy is not the fulfilling computing system associated with the fulfilling pharmacy.

9. A non-transitory machine-readable medium storing instructions that, when executed by at least one processor, cause the at least one processor to perform operations comprising:
receiving, via a communications network communicatively coupled to a network interface, an electronic prescription comprising a prescribed healthcare good associated with a claim for adjudication by a prescription benefits manager (PBM), the network interface including a receiver and a transmitter, the receiver configured to receive the electronic prescription and patient information over the communications network and the transmitter configured to prepare data for transmission over the communications network;
receiving, via the communications network, the patient information electronically from a patient-specific data store communicatively coupled to the communications network, the patient information comprising an identifier linking a patient to a prescription benefit plan and a fulfilling computing system associated with a fulfilling pharmacy, the patient-specific data store configured to store data pertaining to a patient-specific profile;
accessing, via the communications network, a database of pricing data communicatively coupled to the communications network, the database of pricing data stored remotely and accessible via the communications network, the database of pricing data comprising prices of goods at a plurality of participating pharmacies;
selecting a suggested pharmacy accessible to the patient by evaluating the plurality of participating pharmacies at least in part on proximity to the patient and the prices of goods;
sending, via the communications network, the prescription electronically to a computing system of the suggested pharmacy for fulfillment;
determining whether a patient question received from the patient via a user interface is to be directed to at least one of the suggested pharmacy, a professional health services representative, or an insurance benefits manager;
routing, using the network interface having the transmitter and in response to determining the patient question received from the patient via the user interface is to be directed to the insurance benefits manager via the communications network, the patient question electronically to the computing system of the insurance benefits manager;
routing, using the network interface having the transmitter and in response to determining the patient question received from the patient via the user interface is to be directed to the professional health services representative via the communications network, the patient question electronically to the computing system of the professional health services representative; and
routing, using the network interface having the transmitter and in response to determining the patient question received from the patient via the user interface is to be directed to the suggested pharmacy via the network interface, the patient question electronically to the computing system of the suggested pharmacy.

10. The non-transitory machine-readable medium of claim 9, wherein the computing system of the suggested pharmacy is automatically chosen to be a pharmacy associated with the patient.

11. The non-transitory machine-readable medium of claim 9, wherein identifying the suggested pharmacy comprises:
identifying, via the communications network, a plurality of computing systems associated with suggested pharmacy, the plurality of computing systems communicatively coupled to the communications network,
providing, via the communications network, a list of the computing systems associated with the suggested pharmacy to a user in a selectable format, and
receiving, via the communications network, an input from the user indicating a selected computing system of a selected pharmacy, the selected computing system of the selected pharmacy communicatively coupled to the communications network.

12. The non-transitory machine-readable medium of claim 9, wherein the plurality of participating pharmacies participate in a bidding process that includes a first off-cycle bid and a second off-cycle bid.

13. The non-transitory machine-readable medium of claim 12, wherein the first off-cycle bid and the second off-cycle bid are valid for a second period of time that is shorter than a first period of time associated with a regular pricing cycle.

14. The non-transitory machine-readable medium of claim 9, wherein the identifier is a patient-specific identification code or username linking the patient to the patient-specific profile, which stores patient-specific information, including at least one of the prescription benefit plan, a birthdate, a name, and an address.

15. The non-transitory machine-readable medium of claim 9, wherein the patient-specific profile also stores financial account information for the patient.

16. A computer-implemented method comprising:
receiving, using at least one processor via a communications network communicatively coupled to a network interface, an electronic prescription comprising a prescribed healthcare good associated with a claim for adjudication by a prescription benefits manager (PBM), the network interface including a receiver and a transmitter, the receiver configured to receive the electronic prescription and patient information over the communications network and the transmitter configured to prepare data for transmission over the communications network;
receiving, using the at least one processor and via the communications network, the patient information electronically from a patient-specific data store communicatively coupled to the communications network, the patient information comprising an identifier linking a patient to a prescription benefit plan and a fulfilling computing system associated with a fulfilling pharmacy, the patient-specific data store configured to store data pertaining to a patient-specific profile;
accessing, using the at least one processor and via the communications network, a database of pricing data communicatively coupled to the communications network, the database of pricing data stored remotely and accessible via the communications network, the database of pricing data comprising prices of goods at a plurality of participating pharmacies;
selecting, using the at least one processor, a suggested pharmacy accessible to the patient by evaluating the plurality of participating pharmacies at least in part on proximity to the patient and the prices of goods;
sending, using the at least one processor and via the communications network, the prescription electronically to a computing system of the suggested pharmacy for fulfillment;
determining, using the at least one processor, whether a patient question received from the patient via a user interface is to be directed to at least one of the suggested pharmacy, a professional health services representative, or an insurance benefits manager;
routing, using the network interface having the transmitter and in response to determining the patient question received from the patient via the user interface is to be directed to the insurance benefits manager via the communications network, the patient question electronically to the computing system of the insurance benefits manager;
routing, using the network interface having the transmitter and in response to determining the patient question received from the patient via the user interface is to be directed to the professional health services representative via the communications network, the patient question electronically to the computing system of the professional health services representative; and
routing, using the network interface having the transmitter and in response to determining the patient question received from the patient via the user interface is to be directed to the suggested pharmacy via the network interface, the patient question electronically to the computing system of the suggested pharmacy.

17. The method of claim 16, wherein the computing system of the suggested pharmacy is automatically chosen to be a pharmacy associated with the patient.

18. The method of claim 16, wherein identifying the suggested pharmacy comprises:
identifying, via the communications network, a plurality of computing systems associated with suggested pharmacy, the plurality of computing systems communicatively coupled to the communications network,
providing, via the communications network, a list of the computing systems associated with the suggested pharmacy to a user in a selectable format, and
receiving, via the communications network, an input from the user indicating a selected computing system of a selected pharmacy, the selected computing system of the selected pharmacy communicatively coupled to the communications network.

19. The method of claim 16, wherein the plurality of participating pharmacies participate in a bidding process that includes a first off-cycle bid and a second off-cycle bid.

20. The method of claim 19, wherein the first off-cycle bid and the second off-cycle bid are valid for a second period of time that is shorter than a first period of time associated with a regular pricing cycle.
